(19) United States
(12) Patent Application Publication
(10) Pub. No.: US 2009/0047368 A1
(43) Pub. Date: Feb. 19, 2009

(54) Polymeric polyphenol extracted from fermented tea, therapeutic agent for mitochondrial disease, preventive/therapeutic agent for diabetes mellitus, and food or beverage

(75) Inventors: Osamu Numata, Ibaraki (JP); Takashi Fujiwara, Ibaraki (JP); Kazuaki Hosoda, Tokyo (JP); Tetsuo Ozawa, Ibaraki (JP)

Correspondence Address: JORDAN AND HAMBURG LLP, 122 EAST 42ND STREET, SUITE 4000, NEW YORK, NY, 10168, US

(73) Assignee: University Of Tsukuba, Tsukuba-shi (JP)

(21) Appl. No.: 11/667,082

(22) PCT Filed: Nov. 4, 2005
(86) PCT No.: PCT/JP05/20315

§ 371 Date: May 2, 2007

(30) Foreign Application Priority Data
Nov. 4, 2004 (JP) 2004-320980

(51) Int. Cl. A61K 36/82; A61K 31/05; A61P 3/10

(52) U.S. Cl. 424/729; 514/731

(57) ABSTRACT

[Problem] To provide a new pharmacological action for high-molecular-weight polyphenols

[Solution] To provide high-molecular-weight polyphenols extracted from fermented tea (e.g. oolong tea or black tea), which are high-molecular-weight polyphenols with a number average molecular weight ranging from 9,000 to 18,000, and containing in their partial structures a procyanidin structure of polymerized catechins and/or their gallates, and a structure of linkage between the B-rings of catechins and/or their gallates. These high-molecular-weight polyphenols have the functions of mitochondrial activation, suppression of increase in blood glucose, and suppression of weight gain. Therefore, they can be used as drugs for the treatment of mitochondrial disease, and also as drugs for the prevention and treatment of diabetes. They may also be added to health foods and beverages. The selected drawing shows changes in blood glucose level when the said high-molecular-weight polyphenols are given daily to diabetes model mice in specified doses.

### TECHNICAL FIELD

The present invention relates to high-molecular-weight polyphenols extracted from fermented tea having the effect of activating mitochondria; drugs containing these high-molecular-weight polyphenols for the treatment of mitochondrial disease and diabetes; and food and beverages containing the high-molecular-weight polyphenols.

### BACKGROUND ART

Polyphenol is the generic name for compounds that have more than one phenolic hydroxyl group in the same molecule. Polyphenols exist widely in plants. Polyphenols have attracted attention in recent years as they have been found to have various functions, including an anti-oxidant function and an antibacterial function. Currently, many studies are being conducted for the discovery and extraction of various types of polyphenols, as well as the elucidation of their pharmacological actions.

Polyphenols are classified broadly into the families of flavonoids, coumarins, curcumines, lignans, and phenylcarboxylic acids. The flavonoid family is further grouped into catechins, anthocyanidins, flavones, flavonols, isoflavones and flavanes.

Catechins are a group of substances having a flavan-3-ol skeleton containing more than one phenolic hydroxyl group on a C₆—C₃—C₆ skeleton. Tea leaves contain large amounts of catechins. The catechins include, for example, catechin, catechin gallate, epicatechin, epicatechin gallate, epigallocatechin, epigallocatechin gallate, gallocatechin, and gallocatechin gallate. The chemical structure of catechin is shown in Chem. 1 as a representative example. The basic skeleton of the catechins (i.e. catechin and catechin derivatives) consists of A-, B- and C-rings, as shown in Chem. 1. For example, gallocatechin is the result of substituting hydrogen (H) at the 5′ position in the B-ring of the chemical structure shown in Chem. 1 with a hydroxyl group (OH group).

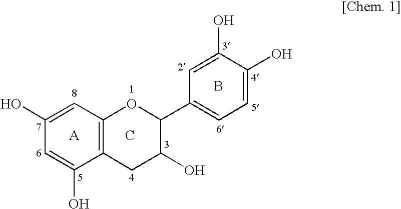

On the other hand, anthocyanins are glycosides having various saccharide components bonded to an aglycone, anthocyanidin (a flavylium compound) comprising a C₆—C₃—C₆ skeleton; they include cyanidin and delphinidin glucosides. They are water-soluble plant pigments found in large amounts mainly in flowers, fruits, and leaves, and are known as antioxidants.

There are highly polymerized types of polyphenols having catechins or anthocyanins linked together (high-molecular-weight polyphenols). High-molecular-weight polyphenols are found in large amounts in wine and fermented tea and are believed to be generated by the polymerization of catechins and anthocyanins during the fermentation and aging processes of wine and tea. Non-Patent Literature 1 contains a description of the chemical structure of high-molecular-weight polyphenols extracted from black tea. Chem. 2 shows the chemical structure of the high-molecular-weight polyphenols contained in Non-Patent Literature 1. The notation R₁ in the chemical structure shown in Chem. 2 represents H (hydrogen) or galloyl, while R₂ and R₃ represent H (hydrogen) or OH (hydroxyl group).

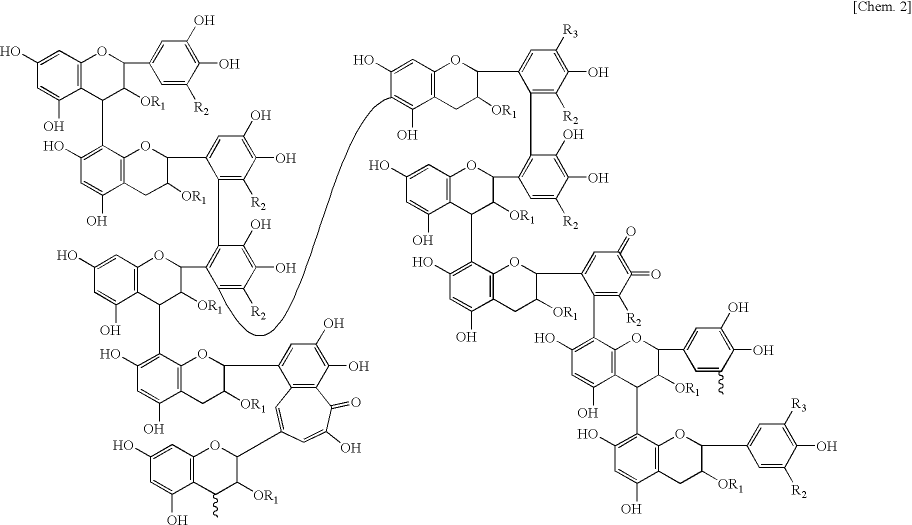

Patent Literature 1 relates to a method for extracting polyphenols. The Description of the Related Art section contains a description of the antioxidative action of polyphenols. Patent Literature 2 relates to the anti-aging effect of catechins and procyanidins on the skin. The anti-aging effect described in this literature is based on the inhibition of activity of MMPs (matrix metalloproteases). Patent Literature 3 relates to a melanin production inhibitor derived from the polyphenols in black tea. The literature states that the inhibitor has a superior effect on the whitening of the skin.

### [Patent Literature 1]

JP H07-196645

### [Patent Literature 2]

JP 2003-252745

### [Patent Literature 3]

JP 2001-158726

### [Non-Patent Literature 1]

Tetsuo Ozawa, Mari Kataoka, Keiko Morikawa, and Osamu Negishi, “Elucidation of the Partial Structure of Polymeric Thearubigins from Black Tea by Chemical Degradation”, Biosci. Biotech. Biochem., 60(12), 2023-2027, 1996

### DISCLOSURE OF THE INVENTION

### Problems to be Solved by the Invention

As mentioned above, polyphenols comprise diverse families. There are many polyphenols which have never been isolated or extracted. In particular, many of the high-molecule-weight polyphenols having highly polymerized or linked catechins, anthocyanins and others have not been isolated or extracted, and their pharmacological action is largely unknown.

Accordingly, the primary objective of the present invention is to isolate and extract new high-molecular-weight polyphenols, and provide new types of pharmacological action for these high-molecular-weight polyphenols.

### Means to Solve the Problem

As the result of diligent study, the inventors of the present invention have discovered that high-molecular-weight polyphenols extracted from fermented tea have the new functions of activating mitochondria, suppressing increases in blood glucose, and suppressing weight gain. Accordingly, the present invention provides high-molecular-weight polyphenols extracted from fermented teas, which are high-molecular-weight polyphenols having an average molecular weight of 9,000 to 18,000, and containing in their partial structures a procyanidin structure of polymerized catechins and/or their gallates, and a structure of catechins and/or their gallates linked by their B-rings.

The high-molecular-weight polyphenols relating to the present invention can be obtained, for example, by first extracting water-soluble components from fermented tea leaves using ethyl acetate, then extracting non-ethyl-acetate-soluble components which could not be extracted in the above ethyl acetate extraction by using butanol, and then by highly purifying the butanol-soluble components, which have been extracted by butanol in the preceding process, using column chromatography with aqueous acetone solvent.

They may also be obtained, for example, by first extracting water-soluble components from fermented tea leaves using ethyl acetate, then extracting non-ethyl-acetate-soluble components which could not be extracted in the above ethyl acetate extraction by using butanol, then acidifying the non-butanol-soluble components which could not be extracted by the above butanol extraction, then performing a second butanol extraction, and finally highly purifying the butanol-soluble components, which have been extracted in the second butanol extraction, using column chromatography with an aqueous acetone solvent.

The high-molecular-weight polyphenols relating to the present invention may contain in their partial structures a catechin structure (shown in Chem. 1 as the structure having the basic skeleton comprising A-, B- and C-rings), a catechin structure to which gallic acid residues are bonded in ester bonds, a procyanidin structure (a structure in which the C-ring of a catechin structure and the A-ring of another catechin structure is linked), a structure in which A-ring of a catechin structure is linked with the B-ring of another catechin structure, a structure in which the B-ring of a catechin structure is linked with the B-ring of another catechin structure, and a quinone structure (and the partial structures may contain an overlapping of these structures).

Since the high-molecular-weight polyphenols relating to the present invention have been found to have the function of activating mitochondria, as described above, mitochondrial disease can be ameliorated using a composition containing these high-molecular-weight polyphenols as a drug.

Additionally, since a mitochondrion, which is an important intracellular organelle whose primary role is ATP synthesis, is responsible for the production of the energy on which cellular activities depend, the activation of mitochondria can enhance cell activity and stabilize cell membranes. Therefore, the use of high-molecular-weight polyphenols can provide the effects of anti-aging action, skin enhancement, promotion of energy metabolism, anti-obesity action, and prevention of anemia in athletes. Furthermore, compositions containing the high-molecular-weight polyphenols relating to the present invention can be applied as a drug to the prevention of anemia in athletes, and also applied to cosmetics. The high-molecular-weight polyphenols relating to the present invention may also be added to food and beverages to produce health foods.

Since the motility of sperm strongly depends on the ability of mitochondria in the flagella to synthesize ATP, the high-molecular-weight polyphenols relating to the present invention can be applied to treatment of male infertility as well as to enhancement of the fertilization rate in the artificial insemination of humans and livestock.

Furthermore, because ciliary motility also strongly depends on the ability of mitochondria to synthesize ATP, the use of the high-molecular-weight polyphenols relating to the present invention can facilitate expectoration and ciliary movement in oviducts. Therefore, a composition containing the high-molecular-weight polyphenols relating to the present invention can be applied for the use as an expectorant and a treatment of female infertility, and the like.

As described above, compositions containing the high-molecular-weight polyphenols relating to the present invention can be applied to pharmaceutical and cosmetic uses. The high-molecular-weight polyphenols relating to the present invention may also be added to health food and beverages.

In addition, since the high-molecular-weight polyphenols relating to the present invention have the effects of suppressing increases in blood glucose and weight gain as described above, they can be applied to the prevention and treatment of diabetes as well as to health foods for the prevention of diabetes.

The terms relating to the present invention are defined below.

The term “fermented tea” means any tea, including oolong tea and black tea, using manufacturing processes which include fermentation.

The term “water” means water (H₂O) as named as a substance. In other words, the term “water” used in the present invention includes boiled water, hot water, and steam. Accordingly, the term “water-soluble components” used in the present invention includes components which elute from fermented tea leaves during extraction using boiled water, hot water, or steam.

The term “mitochondrial disease” means the generic name for various congenital diseases initiated by the genetic mutation of mitochondrial DNA which causes a decrease in mitochondrial function. Mitochondrial disease include ragged-red fiber myopathy, progressive external opthalmoplegia, Leigh syndrome, myoclonus epilepsy with ragged-red fibers (MERRF), mitochondrial myopathy, encephalopathy, lactic acidosis and stroke-like episodes (MELAS), and Leber's optic neuropathy.

### ADVANTAGEOUS EFFECT OF THE INVENTION

The high-molecular-weight polyphenols relating to the present invention are effective in activating intracellular mitochondria.

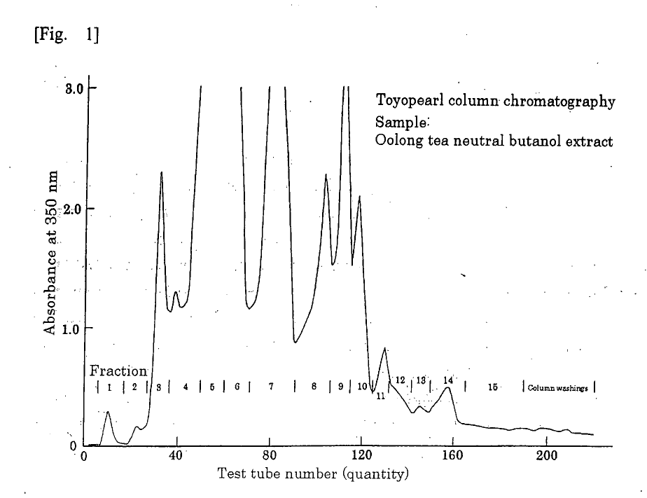

### BRIEF DESCRIPTION OF THE DRAWINGS

[FIG. 1]

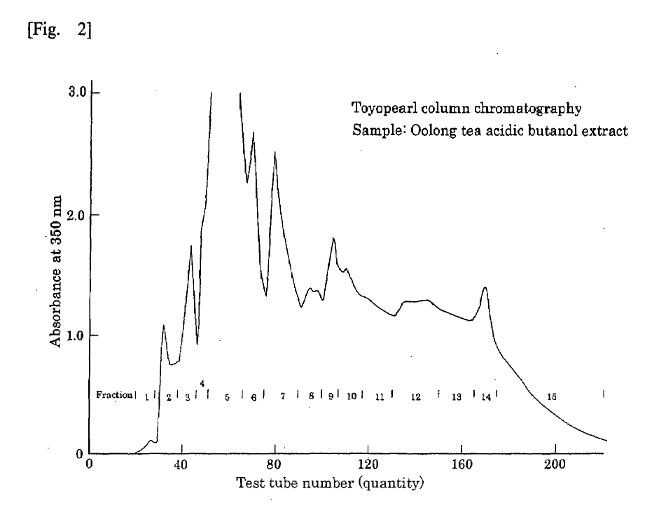

Graph showing the elution curve of neutral butanol extract from oolong tea.

[FIG. 2]

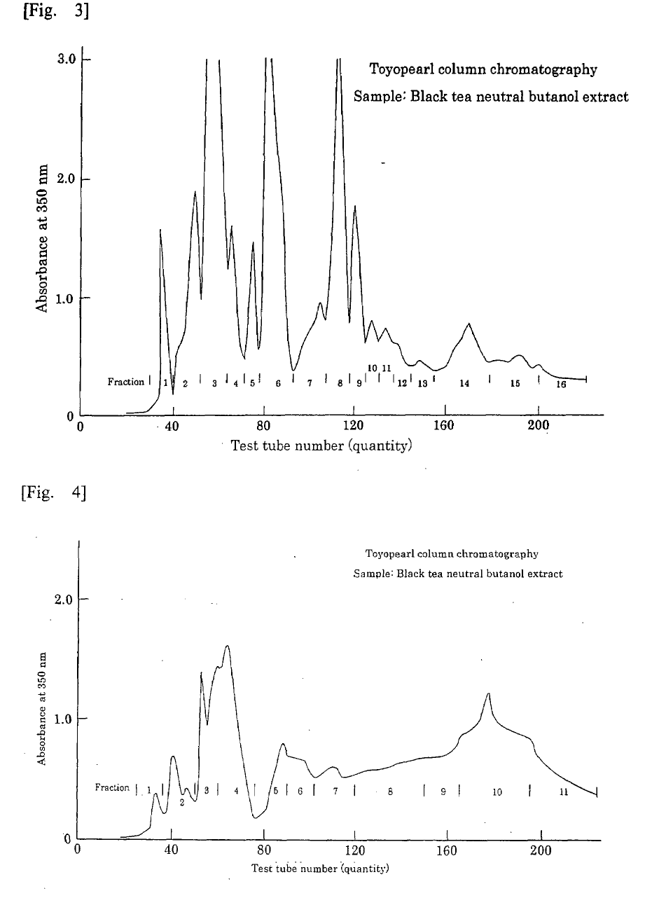

Graph showing the elution curve of acidic butanol extract from oolong tea

[FIG. 3]

Graph showing the elution curve of neutral butanol extract from black tea.

[FIG. 4]

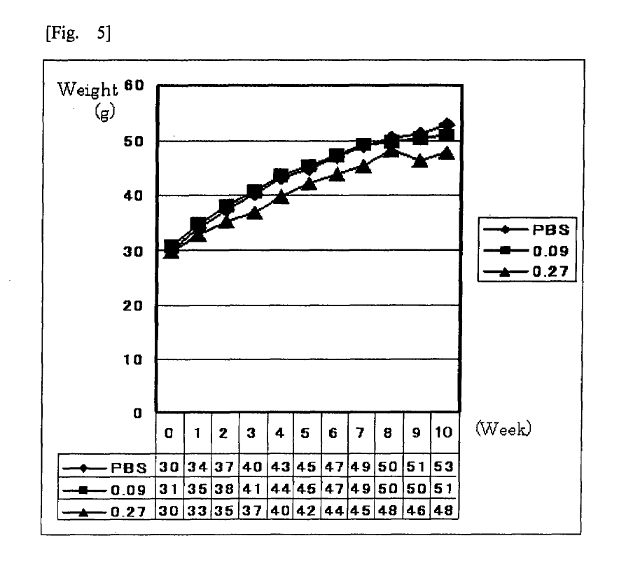

Graph showing the elution curve of acidic butanol extract from black tea.

[FIG. 5]

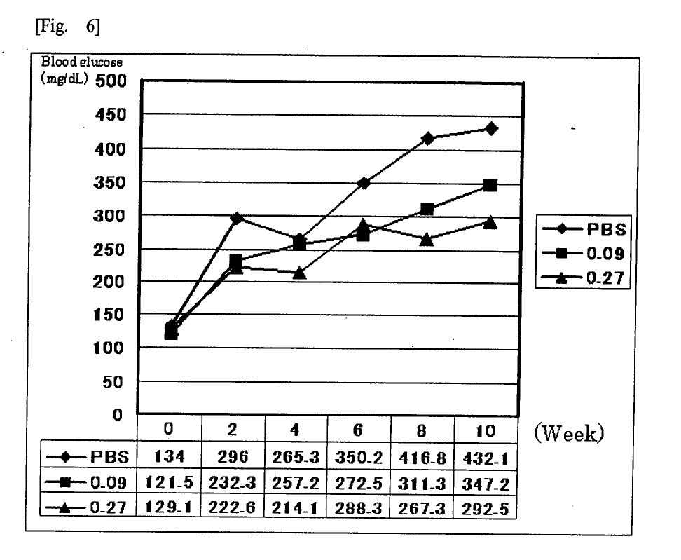

Graph showing changes in body weight of diabetes model mice which have been given the active fraction extracted from fermented tea.

[FIG. 6]

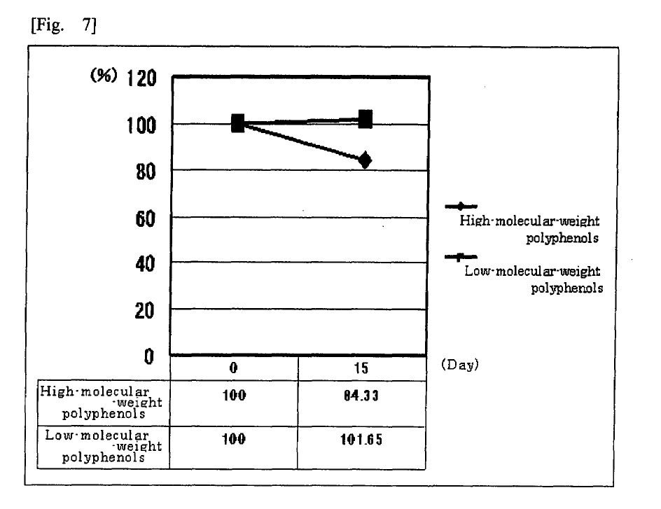

Graph showing changes in blood glucose level of diabetes model mice which have been given the active fraction extracted from fermented tea.

[FIG. 7]

Graph showing changes in blood glucose levels in mice which have been given either high-molecular-weight polyphenols or low-molecular-weight polyphenols.

### EXAMPLE 1

Example 1 relates to the fractionation of the components extracted from fermented tea. For each of oolong tea and black tea, neutral fractions and acidic fractions were extracted using butanol, and further fractionated using column chromatography.

The components of oolong tea were extracted by the following procedures:

First, water-soluble components were extracted from oolong tea leaves by adding 30 g of oolong tea leaves to 1000 mL of boiling water. The mixture was allowed to boil for about 1 minute and stand for 10 minutes afterwards. In the next step, the oolong tea leaves were filtered out to obtain the filtrate. The above process was repeated 4 times to obtain an aqueous solution containing water-soluble components that were extracted from 120 g of oolong tea leaves using hot water.

In the next step, ethyl acetate-soluble components were extracted from the solution containing water-soluble components. This process was performed for the elution and removal by ethyl acetate of relatively low-molecular-weight polyphenols from the water-soluble components of oolong tea. A 200 mL quantity of water-saturated ethyl acetate was added to 500 mL of aqueous solution containing water-soluble components. The mixture was agitated and allowed to stand before the ethyl acetate phase was separated. This process of collecting the separated ethyl acetate phase was repeated 10 times to obtain an extract containing ethyl acetate-soluble components.

The next process produced the “oolong tea neutral butanol extract” by extracting butanol-soluble components from the aqueous phase remaining after the extraction of the ethyl acetate-soluble components. First, the aqueous phase remaining after the ethyl acetate phase was separated in the above-described process for the extraction of ethyl acetate-soluble components was vacuum-concentrated for the removal of residual ethyl acetate. In the next step, 200 mL of water-saturated n-butanol was added to 500 mL of the aqueous phase solution. The solution was agitated and allowed to stand in the same manner as described above, and the butanol phase was separated. This process of collect the separated butanol phase was repeated 10 times to obtain an extract containing butanol-soluble components. Butanol was removed by vacuum concentration of the extract to obtain a solution containing butanol-soluble components. The solution was freeze-dried to become the sample of the “oolong tea neutral butanol extract” (yield: 4.5 g/120 g of oolong tea leaves).

In the next process, the aqueous phase remaining after butanol-soluble components were extracted was acidified to extract n-butanol-soluble components for the second time to obtain the “oolong tea acidic butanol extract”. First, hydrochloric acid was added to the aqueous phase remaining after the butanol phase was separated in the above-described process of extracting the butanol-soluble components, and the pH value was adjusted to about 3. In the next step, 200 mL of water-saturated n-butanol was added to 500 mL of the aqueous phase solution. The mixture was agitated and allowed to stand, and then the butanol phase was separated in the same manner as described above. This process of collecting the separated butanol phase was repeated 5 times to obtain an extract containing butanol-soluble components. In the next step, butanol was removed by the vacuum concentration of the extract to obtain an aqueous solution containing butanol-soluble components. The solution was freeze-dried to become the sample of the “oolong tea acidic butanol extract” (yield: 3.2 g/120 g of oolong tea leaves).

With respect to black tea, components were extracted using the same procedures as for oolong tea, resulting in the production of the “black tea neutral butanol extract” and the “black tea acidic butanol extract”.

In the case of black tea, it should be noted that water-soluble components used as a precursor to the preparation of fractions were extracted by adding 25 g of black tea leaves to 500 mL of boiling water and allowing it to continue boiling gently for 10 minutes, after which leaves of the black tea were immediately filtered out using a Buchner funnel.

This experiment yielded 1.5 g of the black tea neutral butanol extract and 1.9 g of the black tea acidic butanol extract.

In the next process, the above-described extracts extracted by organic solvents were further fractionated by column chromatography. The stationery phase was Toyopearl HW-40F (Tosoh Corporation; Toyopearl is the registered trade mark of Tosoh Corporation), and the mobile phase was an aqueous acetone solution.

First, a column 35 cm long and 2.4 cm in diameter was packed with Toyopearl HW-40F. In addition, 600 mL of 20% acetone solution and 600 mL of 50% acetone solution were prepared as the solvents to be used for the mobile phase.

In the next step, 0.3 g of the above-described organic solvent extracts were dissolved in 3 mL of 20% acetone, and the solution was poured into the column. In the next step, the components of the above-described organic solvent extracts which adsorbed onto the stationary phase (Toyopearl) in the 20% acetone solution were developed with a linear gradient of 20% to 50% acetone using the acetone solutions of 2 different concentrations described above. Then, 5 g aliquots of the eluate were batched off into test tubes using a fraction collector. The flow rate was 0.3 g/minute.

In the next step, the absorbency at 350 nm was measured for each of the eluates collected in the test tubes to develop the elution curves. The organic-solvent extracts were further fractionated based on the elution curves. The eluates in the test tubes belonging to the same fraction were collected, vacuum concentrated to remove acetone, and then freeze-dried.

The components of the fermented tea extracts, which were further fractionated by the procedures described above, were used as samples in the experiments which will be described later in this document.

FIG. 1 shows the elution curve (i.e. the elution pattern) of the oolong tea neutral butanol extract; FIG. 2 shows the elution curve of the oolong tea acidic extract; FIG. 3 shows the elution curve of the black tea neutral butanol extract; and FIG. 4 shows the elution curve of the black tea acidic butanol extract. In FIGS. 1 to 4, the horizontal axes represent the test tube numbers into which the eluates were collected, and the vertical axes represent the absorbency measurements at 350 nm.

The “fractions” indicated in FIGS. 1 through 4 are the fractions which were further fractionated based on the elution curves. As shown in the figures, the oolong tea neutral butanol extract in FIG. 1 and the oolong tea acidic extracts in FIG. 2 were further fractionated as Fractions 1 to 15 respectively, while the black tea neutral extract FIG. 3 was fractionated as Fractions 1 to 16, and the black tea acidic extract in FIG. 4 was fractionated as Fractions 1 to 11.

### EXAMPLE 2

Example 2 examined the mitochondrial activation effect of the fermented tea extract samples, which were fractionated in Example 1.

Mitochondrial activation effect was detected by using rhodamine-123 to measure any increase in mitochondrial membrane potential in protozoa Tetrahymena given fermented tea extract samples. Rhodamine-123 is a reagent which produces a strong fluorescence according to differences in the potential it produces when it binds to the inner mitochondrial membrane and draws out hydrogen ions from the matrix of the mitochondria into the intermembranous area. The experiment used rhodamine-123 manufactured by Sigma Aldrich. The experiment procedures are described below.

First, the samples extracted from fermented teas were given to protozoa Tetrahymena. Each of the fermented tea extract samples fractionated in Example 1 was dissolved in 5% DMSO solution to prepare a 1 mg/mL of fermented tea extract sample solution. A 5% DMSO solution was used as the control. In the next step, for all the sample solutions extracted from fermented tea (including the control solution; the same applies hereafter), 0.3 mL of sample solution (with a final concentration of 0.1 mg/mL) was added to 2.7 mL of Tetrahymena (1-2×10⁴ cells/mL). The solution was shaken for 10 to 12 hours at room temperature. Then, the mixture was centrifuged using a manual rotation centrifuge in order to stop the reaction between Tetrahymena and the fermented tea extract samples. After discarding the supernatant, NKC solution (34.7 mM NaCl, 1.07 mM KCl, 1.08 mM CaCl₂) was added to the mixture to wash away the fermented tea extract samples.

In the next step, a rhodamine-123 stain test was performed. NKC solution and rhodamine-123 (final concentration 10 μg/mL) was added to each of the Tetrahymena samples treated with the respective fermented tea extract samples to make up 3 mL. The stain test was performed after 45 minutes of shaking. Then, the Tetrahymena samples were centrifuged. After discarding the supernatant, the step of adding NKC solution was repeated 7 times to wash away the rhodamine-123.

The next process measured the mitochondrial membrane potential. First, each of the rhodamine-123-stained Tetrahymena samples was shaken for 4 hours to adjust the cell count in each of the samples. In the next step, the rhodamine-123-stained Tetrahymena samples were placed in 96-well plates at 100 μL/well. The samples were allowed to stand for 1 hour. Two types of 96-well plates were prepared: one which was allowed to stand at room temperature for 1 hour and another which was allowed to stand on ice for 1 hour.

After 1 hour of standing, the 96-well plates were set on a microplate reader (excitation: 485 nm; absorption: 535 nm) for the measurements of fluorescence intensity.

The next step calculated the rate of increase in mitochondrial membrane potential. Because the Tetrahymena samples which had been placed on ice for 1 hour had lower motility, the mitochondrial activity remained low regardless of whether or not the samples had any ability for mitochondrial activation. Consequently, the differences between the fluorescence intensities of the at-room-temperature samples and the on-ice samples were calculated, and the resulting values were adopted as the values representing increases in the membrane potential of mitochondria. This experiment calculated the difference in the fluorescence intensities between the at-room-temperature samples and the on-ice samples and converted the values to relative fluorescence intensities using 1 as the difference in intensity of the controls. The relative values were used to represent the rates of increases in the mitochondrial membrane potential.

The results (i.e. the relative values representing the rate of increase in the mitochondrial membrane potential) are shown in Tables 1 to 4. The respective rates of increase in the mitochondrial membrane potential are shown for the oolong tea neutral butanol extract in Table 1, the oolong tea acidic butanol extract in Table 2, the black tea neutral butanol extract in Table 3, and the black tea acidic butanol extract in Table 4. The yields shown in Tables 1 to 4 represent yields from 0.30 to 0.35 g of a fermented tea extract sample recovered after processing through the column, and are the reference values which indicate the content of the high-molecular-weight polyphenol in each of the extracts.

TABLE 1

| | | Membrane potential |
|---|---|---|
| Fraction | Yield (mg) | (relative value) |
| 1 | 6.1 | 4.0 |
| 2 | 14.4 | — |
| 3 | 78.2 | 0.5 |
| 4 | 45.0 | — |
| 5 | 40.9 | 0.4 |
| 6 | 18.2 | 3.3 |
| 7 | 24.1 | 4.2 |
| 8 | — | 4.3 |
| 9 | 13.9 | 7.4 |
| 10 | 13.1 | 10.0 |
| 11 | 16.3 | 10.7 |
| 12 | 8.6 | 17.7 |
| 13 | 12.6 | 19.6 |
| 14 | 16.2 | 25.9 |
| 15 | 12.5 | 32.8 |

TABLE 2

| | | Membrane potential |
|---|---|---|
| Fraction | Yield (mg) | (relative value) |
| 1 | 24.0 | — |
| 2 | 60.5 | −1.0 |
| 3 | 11.0 | 0.4 |
| 4 | 10.1 | 0.8 |
| 5 | 51.5 | 0.8 |
| 6 | 14.6 | 4.8 |
| 7 | 41.8 | 7.3 |
| 8 | 18.2 | 12.0 |
| 9 | 16.0 | 9.2 |
| 10 | 14.5 | 19.7 |
| 11 | 28.8 | 19.1 |
| 12 | 40.9 | 29.6 |
| 13 | 29.2 | 34.3 |
| 14 | 24.4 | 38.6 |
| 15 | 30.5 | 38.2 |

TABLE 3

| | | Membrane potential |
|---|---|---|
| Fraction | Yield (mg) | (relative value) |
| 1 | 70.1 | 1.3 |
| 2 | 21.9 | 1.4 |
| 3 | 40.2 | 2.2 |
| 4 | 7.8 | 4.2 |
| 5 | 7.0 | 5.8 |
| 6 | 22.2 | 2.5 |
| 7 | 18.1 | 5.4 |
| 8 | 17.7 | 8.6 |
| 9 | 7.0 | 12.3 |
| 10 | 3.2 | 16.6 |
| 11 | 10.0 | 18.0 |
| 12 | 11.6 | 18.2 |
| 13 | 14.1 | 20.9 |
| 14 | 25.0 | 22.6 |
| 15 | 16.7 | 18.9 |
| 16 | 10.8 | 25.0 |

TABLE 4

| | | Membrane potential |
|---|---|---|
| Fraction | Yield (mg) | (relative value) |
| 1 | 50.1 | 0 |
| 2 | 17.8 | 0 |
| 3 | 26.6 | −0.21 |
| 4 | 28.2 | 0.31 |
| 5 | 16.0 | 7.8 |
| 6 | 13.9 | 10.8 |
| 7 | 17.1 | 17.6 |
| 8 | 31.6 | 24.5 |
| 9 | 19.0 | 28.7 |
| 10 | 38.6 | 34.9 |
| 11 | 7.7 | 22.5 |

As shown in Tables 1 to 4, all of the neutral and acidic butanol extracts of oolong tea and the neutral and acidic extracts of black tea showed strong increases in membrane potential in the fractions which eluted later in the column chromatography (acetone concentration 35%-50%). In other words, larger increases in mitochondrial membrane potential were detected in the fractions 12 and later for the oolong tea neutral butanol extract (Table 1), in the fractions 10 and later for the oolong tea acidic butanol extract (Table 2), in the fractions 10 and later for the black tea neutral butanol extract (Table 3), and in the fractions 7 and later for the black tea acidic butanol extract (Table 4).

The fractions eluted later in the column chromatography are inferred to be high-molecule-weight polymers having complex polymerization of catechins and/or other polyphenols. Accordingly, it is inferred that the high-molecule-weight polymers having the complex polymerization of catechins and/or other polyphenols increased the mitochondrial membrane potential.

The membrane potential of mitochondria increases when respiratory chain enzyme complexes are active during the ATP production which results in hydrogen ions being drawn out into the intermembranous area. Therefore, these results suggest that the high-molecular-weight polyphenols contained in the fermented tea have the effect of activating the aerobic respiration of mitochondria.

### EXAMPLE 3

Example 3 performed the tannase decomposition of Fraction 15 of the oolong tea acidic butanol extract, which was fractionated in Example 1 (hereafter called the “oolong tea active fraction” in this Example as well as in Example 4 below), and Fraction 15 of the black tea neutral butanol extract, which was also fractionated in Example 1 (hereafter called the “black tea active fraction” in this Example as well as in Example 4 below). Tannase is an enzyme which hydrolyzes gallic acid residues from catechins and tannins.

It should be noted that Fraction 15 of the oolong tea acidic butanol extract was selected from the fractions that had shown the effect of mitochondrial activation in Example 2 to serve as the “oolong tea active fraction”, and therefore it does not necessarily mean that the fractions having mitochondrial activation effects are limited to Fraction 15. (The same conditions apply hereafter). This also applies to the black tea active fraction.

The tannase decomposition was performed using the following procedures. First, 1.10 mg of the oolong tea active fraction and 0.86 mg of the black tea active fraction were dissolved in 0.2 mL of water. To this, 0.3 mL of tannase solution was added, and an enzyme reaction was allowed to take place at 30° C. for 3 hours. The tannase solution was prepared by adjusting tannase (Wako Pure Chemical Industries) to 17.3 U with water.

In the next step, paper chromatography was performed on the enzyme reaction solution (including the decomposition products of the enzyme reaction). The following 2 developing solvents were used for the paper chromatography:

(1) 2% aqueous solution of acetic acid
(2) The top layer of a 4:1:5 (by volume) solvent mixture of n-butanol, acetic acid, and water.

Gallic acid (used as the control) and the enzyme reaction solution were allowed to develop simultaneously on a paper chromatograph, and then sprayed with 0.5% iron alum and 0.5% potassium ferricyanide to allow detection of developed reaction products and obtain Rf values. An Rf (rate of flow) in paper chromatography is the distance from the application point which a developed substance moves in a given time.

Spots were detected at Rf 0.37 when developed by solvent (1) and at Rf 0.59 when developed by solvent (2) for both the oolong tea and black tea active fractions. These spots were in agreement with the Rf value of the gallic acid spot.

Therefore, the results indicate that mitochondria-activating components contained in the oolong tea acidic butanol extract and the black tea neutral butanol extract have, in their high-order structure, the same chemical structures as epicatechin or epigallocatechin, or their gallates.

The inventors carried out similar experiments separately using the oolong tea neutral butanol extract and the black tea acidic butanol extract, which produced similar results. These results indicate that, as described above, the mitochondria-activating components contained in the oolong tea neutral butanol extract and the black tea acidic butanol extract also have, in their high-order structures, the same chemical structure as epicatechin or epigallocatechin, or their gallates.

### EXAMPLE 4

Example 4 performed hydrochloric acid-butanol decomposition of the oolong tea active fraction in the oolong tea acidic butanol extract, which was fractionated in Example 1, as well as the black tea active fraction in the black tea neutral butanol extract, which was also fractionated in Example 1. The procedures were as follows:

A mixed reagent of 1.1 mL hydrochloric acid and 8.9 mL n-butanol was prepared. In the next step, 0.5 mg each of the oolong tea active fraction and the black tea active fraction were respectively placed in small reaction vessels, to each of which 1.0 mL of the above-described mixed reagent was added. The vessels were then sealed with screw caps, placed in a 105° C. autoclave, and heated for 50 minutes.

In the next step, paper chromatography was performed on the reaction products. The following 2 developing solvents were used in the paper chromatography:
(1) Solvent of 30:3:10 (by volume) mixture of acetic acid, hydrochloric acid, and water.
(2) The top layer of a 4:1:5 (by volume) solvent mixture of n-butanol, acetic acid, and water.

Anthocyanin-specific pink spots were detected at Rf 0.57 and Rf 0.36 for the oolong tea active fraction when the chromatograph was developed by solvent (1), and at Rf 0.46 and Rf 0.27 when developed by solvent (2). With respect to the black tea active fraction, anthocyanin-specific pink spots were detected at Rf 0.56 and Rf 0.36 when developed by solvent (1), and Rf 0.46 and Rf 0.27 when developed by solvent (2). Of these spots, those with higher Rf values are believed to represent cyanidin while those with lower Rf values represent delphinidin.

Accordingly, these results indicate that mitochondria-activating components contained in the oolong tea acidic butanol extract and the black tea neutral butanol extract have a procyanidin structure in their high-order structures.

The inventors also carried out similar experiments separately using the oolong tea neutral butanol extract and the black tea acidic butanol extract, which produced similar results. The results indicate that mitochondria-activating components contained in the oolong tea neutral butanol extract and the black tea acidic butanol extract also have a procyanidin structure in their high-order structures, as described above.

To summarize the results of Examples 3 and 4, the components which showed mitochondrial activation in Example 2 are presumed to be high-molecular-weight polyphenols having a high-order chemical structure containing a procyanidin structure of polymerized epicatechins and epigallocatechins and/or their gallates in their partial structures.

### EXAMPLE 5

Example 5 measured the average molecular masses of the active fractions extracted from fermented tea.

The samples used in Example 5 included 4 fractions: Fraction 15 of the oolong tea neutral butanol extract fractionated in Example 1, Fraction 14 of the oolong tea acidic butanol extract also fractionated in Example 1, Fraction 15 of the black tea neutral butanol extract also fractionated in Example 1, and Fraction 11 of the black tea acidic butanol extract also fractionated in Example 1.

Average molecular mass was measured using size-exclusion chromatography (SEC).

As a high performance liquid chromatograph, LC-10A (Shimadzu Corporation) was used with a TSK-GELα-3000 column (column size: 7.8 mm I.D.×30 cm; Tosoh Corporation). The column temperature was set to 40° C. Dimethylformamide containing 10 mM of lithium chloride (LiCl) was used as the developing solvent. The flow rate was set to 0.6 mL/min. The detector used was the UV detector included in LC-10A system. The detection wavelength was set to 275 nm.

First, a molecular mass standard compound was poured into the column to develop a standard curve by plotting the detected UV values on the vertical axis. TSK Standard Polystyrene (Tosho Corporation) was used as the molecular mass standard compound.

In the next step, each of the samples was poured into the column for the measurement of average molecular mass by plotting of the elution times on the horizontal axis and the detected UV values on the vertical axis. The average molecular mass was calculated both in terms of number average molecular weight and the weight average molecular weight.

The results are shown in Table 5.

TABLE 5

| | Number | |
|---|---|---|
| | average | Weight average |
| | molecular | molecular |
| | weight | weight |
| Oolong tea neutral butanol extract (15) | 1.52 × 10⁴ | 2.10 × 10⁴ |
| Oolong tea acidic butanol extract (13) | 1.73 × 10⁴ | 2.44 × 10⁴ |
| Black tea neutral butanol extract (15) | 1.36 × 10⁴ | 1.89 × 10⁴ |
| Black tea acidic butanol extract (11) | 9.43 × 10³ | 1.48 × 10⁴ |

From the results shown in Table 5, the number average molecular weight in the active fractions extracted from fermented tea ranged from 9,000 to 18,000, and the weight average molecular weight from 14,000 to 25,000.

### EXAMPLE 6

Example 6 performed a structural analysis of the active fractions extracted from fermented tea, using a pyrolysis-gas chromatography-mass spectrometry (Py-GC-MS) analyzer.

After pyrolyzing the samples using a pyrolyzer (Py), the decomposition products were separated by introducing them into a gas chromatograph (GC). The separated compounds were analyzed using a mass spectrometer (MS) to obtain thermal characteristics and chemical structure of the sample compounds.

As was the case in Example 5, the samples used in Example 6 included 4 fractions: Fraction 15 of the oolong tea neutral butanol extract fractionated in Example 1, Fraction 14 of the oolong tea acidic butanol extract also fractionated in Example 1, Fraction 15 of the black tea neutral butanol extract also fractionated in Example 1, and Fraction 11 of the black tea acidic butanol extract also fractionated in Example 1.

A Curie point pyrolyzer (JHP-5; Japan Analytical Industry Co., Ltd.) was used for pyrolysis (Py).

First, the temperatures of the interior of the pyrolyzer and the inlet of the gas chromatograph were set to 250° C. In the next step, the samples were wrapped in ferromagnetic pyrofoils (50 μm thick) after adding 5 μL of a 10% solution of tetramethyl ammonium hydroxide in methanol. The samples were then placed in the pyrolyzer. After pyrolytic treatment at 315° C. for 4 seconds, the decomposition products were forwarded to the gas chromatograph. The a 10% solution of tetramethyl ammonium hydroxide in methanol was used to obtain the volatility and the thermal stability during the mass spectrometric stage by methylation of the compounds contained in the samples.

For gas chromatography-mass spectrometry (GC-MS), a gas chromatograph-mass spectrometer JMS-600M (JEOL, Ltd.) was used. For data processing, TSS-2000 (Japan Analytical Industry Co., Ltd.) was used. The gas chromatography used a capillary column HP-1MS (column size: 0.25 mm×30 m; thickness of the coated liquid layer: 0.25 μm; Agilent Technologies).

The pyrolysis products and a carrier gas were introduced into the column to isolate the substances contained in the pyrolysis products and obtain data relating their retention times. Mass spectrometry was performed on each of the isolated substances to obtain data on chemical structures. The temperature inside the column was held initially at 50° C. for 1 minute, and then linearly raised at the rate of 5° C./minute to 300° C., at which it was held for 14 minutes. Helium was used as the carrier gas, with the flow rate set to 1.0 mL/minute. Mass spectrometry was performed with the ion source temperature set at 250° C. and ionization voltage at 70 eV.

The chemical structures of the substances contained in the sample were examined by comparing the data obtained from the gas chromatography-mass spectrometry and the known data for the synthetic standard material.

As the result, 10 compounds were detected in the pyrolysis product of each sample. Chemical structures of these compounds are shown in Chem. 3 to Chem. 5. The retention times (tR) of these compounds are as follows: Compound 1 (tR: 23.0 min.; molecular weight: 168), Compound 2 (tR: 22.1 min.; molecular weight: 166), Compound 3 (tR: 30.3 min.; molecular weight: 196), Compounds 4 (tR: 30.5 min.; molecular weight: 226), Compound 5 (tR: 31.1 min.; molecular weight: 254), Compound 6 (tR: 32.4 min.; molecular weight: 254), Compound 7 (tR: 32.9 min.; molecular weight: 254), Compound 8 (tR: 35.2 min.; molecular weight: 284), Compound 9 (tR: 36.9 min.; molecular weight: 312), and Compound 10 (tR: 46.5 min.; molecular weight: 450).

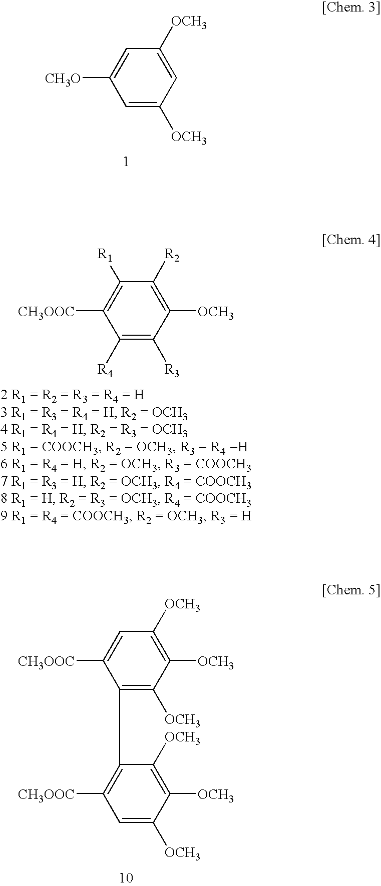

These results indicate that the chemical structures of the compounds contained in the samples include chemical structures which provide the above 10 decomposition products.

In addition, the results of the mass spectrometry suggest that these compounds are polymerized at C2′, C5′, and C6′ positions in the chemical structure of a catechin or its gallate, and on the C4-C8, C6-C6′, or C6′-C6′ linkage between catechins.

To summarize the results of Examples 3 to 6, the components which showed mitochondrial activation in Example 2 are high-molecular-weight polyphenols having a number average molecular weight ranging from 9,000 to 18,000 (a weight average molecular weight ranging 14,000 to 25,000), and having in their partial structures a procyanidin structure of polymerized catechins and/or their gallates and structure of links between the B-rings of catechins and/or their gallates.

Based on the above findings, an example of the high-molecular weight polyphenols relating to the present invention is shown in Chem. 6. It should be noted that the chemical structures of the high-molecular weight polyphenols relating to the present invention are not narrowly limited to the example.

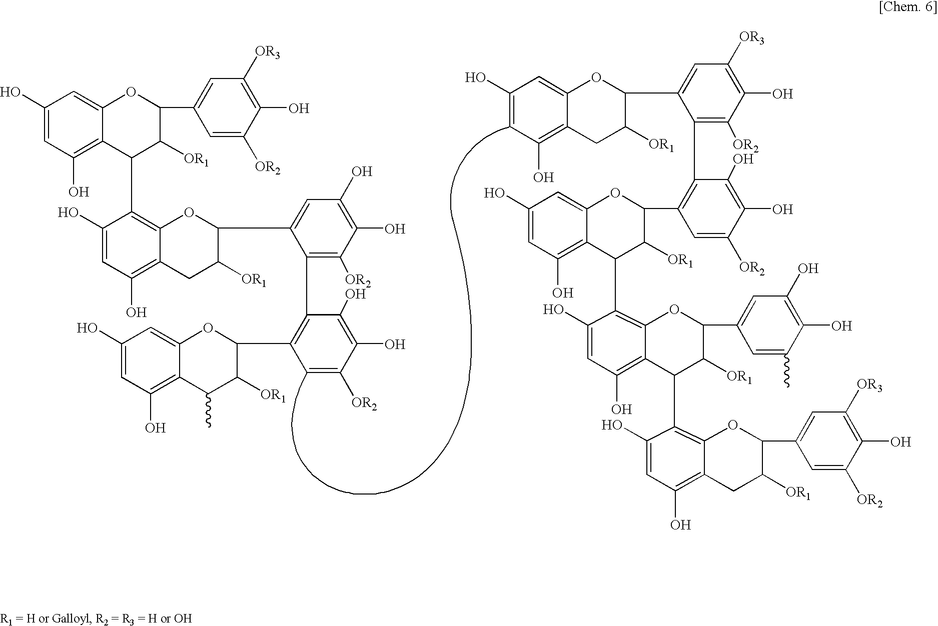

### EXAMPLE 7

Example 7 examined whether the components of the fractions were effective in suppressing increases in blood glucose by administering the active fraction extracted from fermented tea to diabetes model mice.

First, the active fractions obtained in Example 2 were dissolved in PBS for the preparation of doses of 2.7 mg/mL and 0.9 mg/mL. In the next step, the fractions were administered daily by intraperitonal injection to Type II diabetes model mice (BSK. Cg−+Lepr<db>/+Lepr<db>/Jcl, male, 6 weeks of age; purchased from CLEA Japan, Inc.) at the rate of 0.1 mL/day (0.27 mg/day or 0.09 mg/day). The control group was administered 0.1 mL of PBS daily by intraperitonal injection. Blood samples were obtained weekly through the caudal veins for the measurement of blood glucose levels.

The results are shown in FIG. 5 and FIG. 6. FIG. 5 shows changes in body weight and FIG. 6 shows changes in blood glucose levels when the active fraction extracted from fermented tea were given to mice. The horizontal axes in FIG. 5 and FIG. 6 represent the number of weeks from the start of administration. The vertical axes in FIG. 5 and FIG. 6 represent body weight (g) of the mice and blood glucose level (mg/dL), respectively. The number of individuals which were given each fraction ranged from 5 to 6 for each graph.

As shown in FIG. 5, the body weight of the group given the 0.27 mg/day dose was approximately 5 grams less than the group given PBS (control group) after 10 weeks. Also, suppression of the increase in blood glucose levels was also observed after 4 weeks, as shown in FIG. 6, and the levels declined by approximately 33% in comparison with the control group after 10 weeks.

On the other hand, the group given the 0.09 mg/day dose showed a slight suppression of weight gain after 10 weeks in comparison with the group administered PBS (control group), as shown in FIG. 5. As shown in FIG. 6, the suppression of increases in blood glucose levels was also observed after 6 weeks, and the level declined by approximately. 20% after 10 weeks in comparison with the control group.

These results indicate that the administration of relatively high doses of the active fraction extracted from fermented tea can result in early suppression of weight gain and suppression of increase in blood glucose level. Even with a relatively lower dose, weight gain and increase in blood glucose can be controlled by long-term administration.

### EXAMPLE 8

Example 8 used mice to compare the effectiveness between the high-molecular-weight polyphenols relating to the present invention and low-molecular-weight polyphenols in the suppression of increases in blood glucose.

The procedures for the experiment were similar to those employed in Example 7. In this experiment, B6 mice (C57BL; purchased from CLEA Japan, Inc.) were used. The high-molecular-weight polyphenol group was given a daily dose of PBS solution of the active fraction obtained in Example 2 through intraperitonal injection at a dose of 0.27 mg/day. The low-molecular-weight polyphenol group was given a PBS solution of epicatechin daily through intraperitonal injection at a dose of 0.27 mg/day. Blood samples were collected on the first day of the administration and 15 days thereafter for the measurement of blood glucose levels.

The results are shown in FIG. 7. FIG. 7 shows changes in blood glucose levels in mice given either the high-molecular-weight polyphenols or the low-molecular-weight polyphenols. The horizontal axis in the figure represents the number of days from the start of the administration. The vertical axis represents percentage (%) changes in blood glucose based on the blood glucose level on the first day at 100.

As shown in FIG. 7, blood glucose decreased by approximately 16% in the group which was given the high-molecular-weight polyphenols, while there was little change in the group given the low-molecular-weight polyphenols.

The result suggests that the high-molecular-weight polyphenols relating to the present invention have a stronger effect in suppressing increase in blood glucose level than low-molecular-weight polyphenols.

### INDUSTRIAL APPLICABILITY

Compositions containing the high-molecular-weight polyphenols relating to the present invention may be applied to pharmaceuticals and cosmetics. In addition, food and beverages may contain the high-molecular-weight polyphenols relating to the present invention to produce health food.

1.-5. (canceled)

6. A composition, comprising high-molecular-weight polyphenols extracted from fermented tea, said polyphenols having a number average molecular weight ranging from 9,000 to 18,000, and containing in their partial structures a procyanidin structure of linkage between the B-rings of catechins and/or their gallates.

7. A composition according to claim 6, wherein the fermented tea is oolong tea or black tea.

8. A drug or dietary supplement for prevention or treatment of diabetes, comprising a therapeutically effective amount of the composition of claim 6.

9. A drug or dietary supplement for treatment of mitochondrial disease, comprising a therapeutically effective amount of the composition of claim 6.

10. A food or beverage for prevention or treatment of diabetes, comprising a therapeutically effective amount of the composition of claim 6.

11. A food or beverage for treatment of mitochondrial disease, comprising a therapeutically effective amount of the composition of claim 6.

12. A method of preventing or treating diabetes, comprising administering the dietary supplement or drug of claim 8.

13. A method of treating mitochondrial disease, comprising administering the dietary supplement or drug of claim 9.

14. A method of preventing or treating diabetes, comprising ingesting the food or beverage of claim 10.
